(19) United States
(12) Patent Application Publication
(10) Pub. No.: US 2009/0012049 A1
(43) Pub. Date: Jan. 8, 2009

(54) Use of an Unsaponifiable Extract of Plant Pulp in the Treatment of Skin Ageing.

(76) Inventors: Bernard Fabre, Belderaud (FR); Rene Belle, Saix (FR); Marie Charveron, Toulouse (FR); Caroline Baudouin, Rambouillet (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(21) Appl. No.: 12/087,390

(22) PCT Filed: Dec. 28, 2006
(86) PCT No.: PCT/FR2006/002908

§ 371 Date: Jul. 1, 2008

(30) Foreign Application Priority Data
Jan. 5, 2006 (FR) 06 00077

(51) Int. Cl. A61K 31/56; A61K 8/33; A61P 17/00

(52) U.S. Cl. 514/170

(57) ABSTRACT

The field of the present invention relates to the use of an unsaponifiable extract of plant pulp in the preparation of a cosmetic, pharmaceutical or nutraceutical product intended for treating and/or preventing skin disorders associated with ageing.

The field of the present invention relates to the use of an unsaponifiable extract of plant pulp in the preparation of a cosmetic, pharmaceutical or nutraceutical product intended for treating and/or preventing skin disorders associated with ageing.

Ageing is an inevitable, slowly progressive, and irreversible phenomenon causing anatomical and histological changes that are responsible for functional anomalies of the organs. The first visible signs show themselves in skin tissue by changes in texture, color and transparency and by the appearance of wrinkles. These signs can be potentiated by extrinsic factors such as sun, tobacco etc.

The importance of oxygen free radicals (OFRs) in the processes involved in ageing is taken as being one of the main theories.

In the skin, OFRs have been described as early mediators of inflammatory pathologies and ageing (Kress M. et al. Pain 1995; 62:87-94).

In the course of ageing, all the structures of the skin undergo changes. However, the principal changes are concentrated in the dermis, the fibroblasts and extracellular matrix being the main targets and the main players. The fibroblasts are capable of entering senescence. As a consequence, their number decreases, their function is slowed and their phenotype is modified. They then play an active part in the degradation of the extracellular matrix of the dermis. In addition, during senescence, the fibroblasts lose their reactivity and see their regulation modulated. It is in fact recognized that ageing is associated with a reduction in, or even loss of, the response to environmental stress and, as a result, is associated with the appearance of infectious and autoimmune diseases and cancers (Gardner I. D. Rev. Infect. Dis. 1980; 2: 801-10). The appearance of wrinkles is one of the first signs of ageing. For some people this constitutes a real problem in relations with their exterior. Accordingly, numerous cosmetic products aimed at treating skin ageing are now available to the public. In the main, those specialties are based on plant extracts.

The Argan tree, known in the international botanical nomenclature by the name Argania spinosa (L.) Skells, and more particularly the seed kernel, has been utilized especially by the cosmetics industry.

The Argan is a squat tree, 6 to 10 meters tall, having a habit which is reminiscent of that of the Olive.

The habit of the leaf crown is quite variable, and may be upright or weeping. The branches, which are very thorny, bear very small, short (approximately 2 cm), slender, alternate, lanceolate leaves, often grouped into fascicles.

The Argan is usually evergreen but in very dry periods it may lose its leaves.

The flowers, which are yellow-green in color, hermaphrodite (stamens and pistils on the same flower) and pentamerous (5 petals, 5 sepals etc.), are grouped into inflorescences of the glomerular type. They bloom from May to June.

The Argan starts to bear fruit from the age of 5 years onwards. The fruit is an oval, yellow, sessile berry from 4 to 5 cm in length. It is formed of a fleshy pericarp (also called the pulp) containing a kind of very hard, brown-colored “false stone”. The latter is actually composed of 2 or 3 joined-together flattened seeds, each of which contains an oleaginous kernel.

The most utilized applications are based on the seed kernel, which yields an oil and, subsequently, an oil-cake.

A number of patents for inventions relate to the oil derived from the seeds: obtaining the oil with solvent (FR 2 553 788); argan oil enriched in unsaponifiable components (FR 2 724 663).

Substances other than the oil have also been patented, this being the case with peptides derived from the seed oil-cakes obtained after extraction of the oil: association of oil and peptides of oil-cakes for the treatment of disorders related to skin ageing (FR 2 756 183). The leaf of the Argan and the proteins and saponins of oil-cakes have also been the subject of patents for inventions: EP 1 213 025 relates to leaf extracts, EP 1 213 024 deals with proteins of oil-cakes and EP 1 430 900 with saponins of oil-cakes.

More recently, the pulp of fruit of the Argan has been the subject of the Patent Application WO2005/039610.

The fruit of the Argan is a false drupe. It is accordingly composed of a fleshy pericarp called the pulp (55 to 75% of the fruit) and a stone provided with a very hard shell containing from one to three kernels. The oil is extracted from the kernels.

The pulp of the fruit has been subject to chemical tests. It is composed of carbohydrates, including cellulose, glucose, fructose and sucrose (Charrouf Z. Guillaume D., Ethnoeconomical, ethnomedical, and phytochemical study of Argania spinosa (L.) Skeels., Journal of Ethnopharmacology, 1999, 67, 1, 7-14—Sandret F. G., Etudes préliminaires des glucides et du latex de la pulpe du fruit d' Argan (Argania spinosa): variation au cours de la maturation, Bulletin de la Société de Chimie Biologique, 1957, 39, 5-6, 619-631). Lipids are also present therein. Their content is 6%. In the unsaponifiable fraction of those lipids there have been identified 5 triterpene alcohols=erythrodiol, lupeol, α- and β-amyrine and betulinaldehyde, and 2 sterols=α-spinosterol and schottenol (Charrouf Z., Fkih-Tetouani S., Charrouf M., Mouchel B., Tritérpenes et stérols extraits de la pulpe d'Argania spinosa, Plantes Médicinales et Phytothérapie, 1991, 25, 2-3, 112-117).

The Patent Application WO2005/039610 relates in a general manner to the use of a composition based on pulp of fruit of the argan tree in the preparation of cosmetic products. The extract of the fruit pulp was more or less purified. The inventors did in fact test the extract at various stages in the process. Accordingly, it is the use of an extract of fruit pulp obtained following extraction with hexane that is preferentially described (page 15). Then, following a conventional saponification step known to the person skilled in the art, the authors tested the unsaponifiable fraction collected in the process. Finally, the authors also envisaged a step of fractionation of the unsaponifiable component by chromatography, taking care to remove the triterpene compound erythrodiol.

The reasoning for this was probably guided by the results obtained, especially by the fact that erythrodiol by itself is shown as being toxic (Example 1) at a dose which is lower than the triterpene fraction as defined in the document: fraction A devoid of erythrodiol (page 38). In addition, erythrodiol by itself is of only mediocre benefit with respect to UVA and UVB (Examples 3 and 4), compared to said triterpene fraction. The general teaching of that document accordingly concerns the use of the triterpene fraction of an extract of pulp of the fruit of the argan tree in the preparation of cosmetic products and preferably in the treatment of skin damaged by UVA and UVB via stimulation of the metabolism of fibroblasts. More specifically, this document teaches that said triterpene fraction, as disclosed in WO2005/039610, will be all the more active the lower the amount of erythrodiol.

In a surprising and unexpected manner, the authors of the present invention have established an inhibitory effect on the senescence of fibroblasts of mature skin due to an unsaponifiable extract of pulp of fruit of the argan tree which is rich in erythrodiol, it being possible to obtain said extract by acetone extraction followed by a conventional saponification step. However, it is reasonable to expect that the benefits of the present invention may be extended to any unsaponifiable extract of plant pulp which has a triterpene fraction the composition of which, in terms of its major components, is close to that derived from pulp of the fruit of the argan tree.

The present invention relates to the use of an unsaponifiable extract of plant pulp comprising a triterpene fraction, characterized in that said triterpene fraction comprises erythrodiol, α-amyrine, β-amyrine and lupeol, in the preparation of a cosmetic, pharmaceutical or nutraceutical product intended for preventing and/or treating disorders of the skin associated with skin ageing. Preferably, said extract is obtained by acetone extraction followed by a conventional saponification step.

This unsaponifiable extract, also called the initial unsaponifiable component, may be dissolved in an excipient in order to facilitate its formulation.

Preferably, said extract is obtained starting from a plant selected from the Sapotaceae family; and, even more preferably, said extract is obtained starting from pulp of fruit of the argan tree.

One advantage of acetone extraction lies in the fact that it is possible for the latex, which represents the very great majority of the lipid fraction, to be removed and accordingly for the lipid fraction to have a higher concentration of unsaponifiable substances.

The composition of the unsaponifiable component according to the present invention differs both qualitatively and quantitatively from that preferentially described in the Patent Application WO2005/039610.

The extracts according to the present invention are characterized by their content of triterpene substances. The latter can be analyzed by gas-phase chromatography according to a suitable conventional method allowing β-amyrine and erythrodiol to be identified. In contrast, α-amyrine and lupeol are not separated by this method, so that these compounds can be assayed jointly.

Advantageously, the triterpene fraction of said extract is composed of erythrodiol the mass fraction of which is between about 7% and about 40%, inclusive, of the initial unsaponifiable component, β-amyrine the mass fraction of which is between about 5% and about 30%, inclusive, of the initial unsaponifiable component, and α-amyrine and lupeol the sum of the two mass fractions of which is between about 10% and about 50%, inclusive, of the initial unsaponifiable component.

Said mass fraction of erythrodiol is advantageously between about 10% and about 20%, inclusive, of the initial unsaponifiable component and, even more advantageously, is equal to about 15% of the initial unsaponifiable component.

Said mass fraction of β-amyrine is advantageously between about 7% and about 20%, inclusive, of the initial unsaponifiable component and, even more advantageously, is equal to about 10% of the initial unsaponiflable component.

The sum of said mass fractions of α-amyrine and lupeol is advantageously between about 15% and about 30%, inclusive, of the initial unsaponifiable component and, even more advantageously, is equal to about 20% of the initial unsaponifiable component. The contents of these various compounds depend on the extraction conditions. These values will be lower in the cosmetic, pharmaceutical or nutraceutical product as a function of the excipient or excipients that will be added to the initial unsaponifiable component.

A notable point of the present invention is the important contribution that the erythrodiol makes to the anti-ageing properties of the unsaponifiable extract according to the present invention. As OFRs play an important part in the process of skin ageing, the anti-free-radical (anti-OFR) effect of erythrodiol was assessed compared to the unsaponifiable component of pulp of fruit of the argan tree according to the present invention.

The damage caused by OFRs within cells is reflected by changes in the lipid components of the plasma membrane (lipoperoxidation), proteins (denaturation and degradation) and genetic material or DNA (mutations). The tests carried out in vitro related to determination:
of the efficacy of protection afforded by erythrodiol and by the unsaponifiable extract against oxidation of membrane lipids (Example 3);
and of the protective power of erythrodiol and of other triterpene compounds (lupeol, α- and β-amyrine) with respect to degradation of genomic DNA (Example 4). In these tests it was possible to demonstrate that erythrodiol is a compound which has considerable antioxidant potential.

In a particular embodiment of the present invention, the extraction can be carried out as follows: the dried pulp of fruit of the Argan tree is crushed and then extracted with acetone. It is also possible to use an acetone/water mixture. The extraction is performed with stirring or statically, in a plant:solvent ratio that may vary from 1:2 to 1:20, at temperatures varying from ambient temperature to the boiling point of the solvent and over a period that may range from 30 minutes to 24 hours.

Once extracted, the solid plant residue is separated from the extractive solution by filtration or centrifugation. The solution may be concentrated to a greater or lesser extent, as far as production of a dry extract. In this latter case, the dry material may be dissolved in an alcohol to allow saponification.

To the solution there is added a metal hydroxide, in particular sodium hydroxide or potassium hydroxide at concentrations varying from 0.1N to 10N. The saponification is performed at temperatures ranging from ambient temperature to boiling, with stirring, and over a period ranging from 15 minutes to 48 hours depending on the temperature. Purification is performed by liquid/liquid extraction. To the hydrolysis medium there is then added a non-miscible solvent which may be water which is saturated or not with salts [NaCl, (NH₄)₂SO₄] and adjusted to a pH ranging from 3 to 9. This solvent may be an ether oxide, ester, alkane, halogenated hydrocarbon, or a mixture of those solvents. One, two or three successive liquid/liquid extractions are carried out. The organic phases are combined and then washed with water saturated or not with salts and at a pH ranging from 3 to 9. This washing phase may be repeated several times.

After purification, the organic phases are treated so as to remove the solvent. This treatment can be carried out by evaporation whilst controlling the pressure. The evaporation step may result in a product of more or less waxy, fatty consistency, the initial unsaponifiable component.

There may be added an excipient which may be an animal wax (beeswax, for example) or vegetable wax (Carnauba wax, Candellila wax or Jojoba wax), a vegetable oil (maize, carthame, sesame, argan etc.), glycerin, products of synthetic origin such as vaseline oil, polyols (such as propylene glycol, butylene glycol, glycerol etc.), esterified triglycerides (such as miglyol 812, myritol 318, neobee MJ), oxypropylenated polymers of formula H(OCH₂—CHCH₃)ₙ OH or oxyethylenated polymers of formula H(OCH₂—CH₂)ₙ OH, diesters of fatty alcohols of variable length, from C₁ to C₄₀. The proportions of the initial unsaponifiable component of pulp of fruit of the Argan tree and the excipient can vary from 1/99 to 99/1.

Advantageously, the present invention allows utilization of the fruit pulp in anti-ageing treatment, at a reasonable cost price. The unsaponifiable extract is used without an additional purification step, which is very costly. The composition according to the present invention can accordingly be obtained using a process which involves conventional extraction and saponification steps known to the person skilled in the art.

The use of an unsaponifiable extract of plant pulp according to the present invention makes it possible to prevent and/or treat skin disorders which are made manifest by changes in the texture, color and transparency of the skin and by the appearance of wrinkles.

In a particular embodiment of the invention, the skin disorders are due to a reduction in, or loss of, response to environmental stress, especially caused by the sun or tobacco. In another particular embodiment of the invention, the skin disorders are due to a reduction in, or loss of, inducibility of HSP72 proteins. HSP (standing for “Heat Shock Protein”) proteins are expressed constitutively in numerous cells and have functions that are indispensable for the maintenance of proteins, hence their name “chaperone” proteins. In fact they inhibit the aggregation of denatured proteins, prevent inappropriate associations of proteins and are involved in intracellular transport and in maintaining certain proteins in inactive form (Morris S. D. Clin. Exp. Dermatol. 2002; 27: 220-224). HSPs also play an essential part in the response to stress and especially in cellular protection processes bringing into play the adaptive response (Maytin E. V. J. Invest. Dermatol. 1995; 104: 448-55).

In unexpected manner, the use of an extract according to the present invention makes it possible to restore induction of HSP72 proteins in senescent fibroblasts.

Within the context of the present invention, the cosmetic, pharmaceutical or nutraceutical product comprising an extract according to the invention is administered by the oral route or topical route, preferably by the topical route.

For administration by the topical route, the galenical form is selected from the group comprising creams, gels, ointments and sprays.

Advantageously, the oral form is selected from the group comprising tablets, capsules and powders for drinkable suspensions.

Advantageously, the amount of said extract in the final cosmetic product is between about 0.001% and about 50% inclusive, preferably between about 0.01% and about 10% inclusive and even more preferably between about 0.1% and about 2% inclusive by weight of the total weight of the preparation.

The preparation may additionally comprise other active substances known to the person skilled in the art for the treatment and/or prevention of skin disorders associated with skin ageing. Advantageously, said preparation contains other substances obtained from the argan tree that are known for their “anti-ageing” action such as, for example, the oil obtained from the kernel of the seed and the peptides of oil-cakes.

The example hereinbelow of a composition according to the invention is given by way of non-limiting example. The percentages are given by weight, with respect to the total weight of the composition.

### EXAMPLE 1

### Anti-Sag Face Treatment

| | |
|---|---|
| Unsaponifiable extract of pulp of fruit of the argan tree | 0.1 to 2% |
| Enriched argan oil | 1 to 5% |
| Argan peptides | 0.1 to 1% |
| Vitamin E derivative | 0.1 to 0.5% |
| Vitamin F glyceric ester | 0.1 to 0.5% |
| Vitamin A palmitate | 0.1 to 1% |
| Methyl glucose stearate | 1 to 5% |
| Capric/caprylic triglycerides | 2 to 8% |
| Liquid paraffin | 5 to 12% |
| Parfum | q.s. |
| Purified water | q.s.p. 100 g |

The following Examples illustrate the present invention without, however, limiting its scope.

### EXAMPLE 2

### Method of Obtaining an Unsaponifiable Extract of Pulp of Fruit of the Argan Tree

1 tonne of dried pulp of fruit of the Argan tree is crushed and then extracted in a reactor with 5 tonnes of acetone. The extraction is stirred for one hour at reflux. Once cooled, the solution is recovered by filtration and then concentrated in vacuo until a desolvented oily extract is obtained. This residue is taken up in 500 liters of ethanol 95% v/v. 100 liters of 10N sodium hydroxide solution are added thereto and refluxing is carried out, with stirring, for one hour.

After cooling, the hydrolyzed solution is placed in a separator and 500 liters of heptane and 300 liters of water are added thereto. Liquid/liquid extraction is carried out with care. After separating, the organic phase is collected. 2 further extractions are conducted using 500 liters of heptane. The 3 heptane phases are combined and washed 3 times using 500 liters of water each time. The washed organic phases are desolvented. A waxy paste is thereby obtained. This extract, which corresponds to the initial unsaponifiable component, is assayed for its content of triterpene substances. It contains 10% β-amyrine, 15% erythrodiol and 20% of the mixture lupeol/α-amyrine.

### EXAMPLE 3

### Analysis of the Anti-Free-Radical Effect of Erythrodiol—Analysis of Lipid Peroxidation

### 1) Introduction

The plasma membrane constitutes the first and main target of OFRs and, being rich in lipids, it is the site of increased peroxidation (Girotti A. W. J. Free Radic. Biol. Med. 1985; 1: 87-95). The peroxides generated in the course of that lipid oxidation are also highly reactive and capable of degrading protein material and genomic material.

In order to assess membrane degradation, the authors of the invention have measured lipid peroxidation by means of an in vitro assay of complexes formed between the products of lipid oxidation and thiobarbituric acid. These complexes are called TBARS (standing for “Thiobarbituric Acid Reactive Substances”) and give their name to the test: the TBARS Test.

In order to mimic chemical oxidative stress, the fibroblast line L929 was treated with a complex composed of hydrogen peroxide (H₂O₂) and iron (Fe²⁺/Fe³⁺), accordingly reproducing Fenton's reaction, a source of OFRs and, more particularly, the free hydroxyl radical (OH^{∘}) (Vessey D. A. et al J. Invest Dermatol. 1992; 99: 859-63): H₂O₂+Fe²⁺→OH^{∘}+OH⁻+Fe³⁺.

### 2) Methodology

Products Tested:

The products were assessed using the murine fibroblast line L929. The cells are pre-treated with the various concentrations of products (Table I) for 16 hours and then stimulated with the H₂O₂—Fe²⁺/Fe³⁺ complex for 1 hour. The LK0304 batch of unsaponifiable extract of pulp of fruit of the argan tree was prepared in accordance with Example 2.

TABLE I
Compilation of solutions tested

| Reference product | Mother solution | Solutions tested |
|---|---|---|
| Unsaponifiable component of | 10 mg/ml | 0.3 μg/ml |
| pulp of fruit of the argan tree | (DMEM/ | 1 μg/ml |
| Batch: LK0304 | TWEEN20) | 3 μg/ml |
| | −20° C. | |
| Erythrodiol/ | 10 mg/ml DMSO | 0.3 μg/ml-0.68 μM |
| EXTRASYNTHESE | −20° C. | 1 μg/ml-2.26 μM |
| Batch: 05040605 | | 3 μg/ml-6.78 μM |
| Vitamin E* SIGMA T-1539 | 400 mg/ml | 400 μg/ml-928.7 μM |
| | −20° C. | |

(*Reference anti-free-radical compound)

The peroxidation of membrane lipids is analyzed by TBARS measurement (according to Morliére P. et al Biochim. Biophys. Acta. 1991; 1084: 261-268).

Principle of the Test:

In an acid medium, at 95° C., complexes referred to as TBARS (standing for “ThioBarbituric Acid Reactive Substances”) are formed between the lipid oxidation products (malondialdehyde or MDA) and thiobarbituric acid (TBA), which can be fluorescence-assayed relative to a MDA standard series. The TBARS assay value is then expressed in pmol/μg of proteins. The proteins and TBARS are assayed in the intracellular medium.

Calculation of the Percentage Protection of Cell Membranes:

Starting from the calculation of the TBARS in pmol/μg of proteins, the protective efficacy of the various products against oxidation of the lipid membranes was calculated as follows.

$$\% protection = \frac{[ T B A R S control ] - [ T B A R S ( + products ) ]}{[ T B A R S control ]} \times 100$$

### 3) Results—Discussion

After treatment for 16 hours with the various products being tested, the free-radical stress model used in this experiment (Fenton's reaction) causes substantial lipid peroxidation in the L929 fibroblasts. This massive discharge of the free hydroxyl radical OH^{∘} accordingly generates oxidative stress at the cellular level and especially at the level of the membranes. However, in this type of oxidative reaction, the products produced by the lipid peroxidation are internalized into the cells and the TBARS are therefore assayed in the intracellular medium.

The results obtained are compiled in Table II below.

TABLE II
Lipid peroxidation analysis

| | Protection of membrane lipids in % | | |
|---|---|---|---|
| | | Standard | N: number of |
| Active substances tested | Mean | deviation | experiments |
| Vit E 400 μg/ml-928.7 μM | 56.62 | 8.74 | 3 |
| Erythrodiol | | | |
| 0.3 μg/ml-0.68 μM | 33.75 | 15.39 | 3 |
| 1 μg/ml-2.26 μM | 38.43 | 7.68 | 3 |
| 3 μg/ml-6.78 μM | 53.22 | 17.30 | 3 |
| Unsaponifiable component of | | | |
| pulp of fruit of the argan tree | | | |
| 0.3 μg/ml | 21.38 | 37.53 | 3 |
| 1 μg/ml | 30.53 | 17.80 | 3 |
| 3 μg/ml | 37.39 | 9.45 | 2 |

Vitamin E, which represents the anti-free-radical reference compound, reduces the lipid peroxidation caused by the complex H₂O₂—Fe²⁺/Fe³⁺ and very effectively protects the cell membranes (about 56%).

The unsaponifiable extract of pulp of fruit of the argan tree prepared according to Example 2 has an anti-free-radical activity at concentrations of 1 and 3 μg/ml (30% and 37% protection of lipid membranes, respectively).

Erythrodiol, a compound contained in the triterpene fraction of the unsaponifiable extract, has good anti-oxidant activity, with a dose-dependent effect. Erythrodiol is active from 0.3 μg/ml (33% protection). The anti-free-radical effect of erythrodiol at 3 μg/ml is very substantial and comparable to Vitamin E.

### 4) Conclusion

The in vitro model presented in this study reflects the consequences due to major oxidative stress on the main cellular target which is the plasma membrane. The lipid peroxidation assay value accordingly constitutes a good marker of the oxidative stress and allows assessment of the anti-oxidant action, with respect to the hydroxyl radical, of active substances at the level of the cell membrane.

Vitamin E, an anti-oxidant compound, allows validation of this model.

Under these test conditions, it was observed that the extract according to the present invention, which comprises erythrodiol, and also erythrodiol itself have substantial anti-oxidant potential.

### EXAMPLE 4

### Analysis of the Anti-Free-Radical Effect of Erythrodiol—Analysis of Genomic Damage

### 1) Introduction

DNA is a target of OFRs, which cause base modifications (oxidation, nitration, deamination: Guetens G. et al Clin. Lab. Sci. 2002; 39: 331-457), formation of strand breaks (abasic sites or β-elimination) and DNA-protein or DNA-hydroperoxide cross-linking. The degradation of the genomic material causes a cascade of cellular reactions (replication fork blockage, activation of key proteins, arrest in the cell cycle) which in the end bring about repair mechanisms. The bases modified by oxidative stress are accordingly taken charge of in the main by the base repair system or BER, standing for “Base Excision Repair” (Friedberg E. C. et al DNA repair and mutagenesis, ASMPress; Washington D.C. 1995). This system acts rapidly and efficiently by way of three key steps:
1—recognition of the damaged base;
2—incision and excision of the lesion;
3—resynthesis of the breach.

OFRs can be produced in such an amount that the cellular defense and repair systems can be saturated. If apoptosis effectors are activated, the damaged cell dies. However, should the DNA lesions be badly repaired, harmful mutations can be generated which then play a part in the initiation step of carcinogenesis. This is why the biological effect of oxidative stress (mortality or mutagenesis) underlies longer-term events such as ageing and cancer.

Numerous studies have demonstrated the strong correlation between ageing and the progressive and irreversible accumulation of oxidative damage at the level of cellular macromolecules. Several research groups have shown in rodents that the levels of 8-OxoGuanine, measured in various tissues such as the skin, increase with age (Tahara S. et al Mech. Ageing Dev. 2001; 122: 415-426). The work by Mecocci P. et al (Free Radic. Biol. Med. 1999; 26: 303-8) on skeletal muscle in humans shows that oxidative lesions to DNA or to lipids accumulate with age. The same team has also shown that, in subjects suffering from Alzheimer's disease, the levels of oxidized bases in the DNA of lymphocytes and the levels of anti-oxidants in plasma are significantly higher and lower, respectively, than in healthy subjects (Mecocci P. et al Arch. Neurol. 2002; 59: 794-8).

### 2) Objective

Following on from Example 3 and in order to check the anti-free-radical activity of erythrodiol in another model, the authors of the invention have analyzed its protective power with respect to genomic DNA damage caused by oxidative stress, in comparison with the unsaponifiable extract of pulp of fruit of the argan tree and other triterpene compounds also contained in said extract.

They decided to generate the lesions to DNA by means of H₂O₂ stress and to analyze indirectly the damage thereby caused, by analyzing the repair reaction. For that purpose, a kit referred to as the 3D (standing for “DNA Damage Detection”) test was used. This biochemical test mimics, in vitro, the repair reaction by means of excision (Salles B. et al Anal. Biochem. 1995; 232: 37-42 and Salles B. et al Biochimie 1999; 81: 53-58). The 3D test is based on the repairing of DNA lesions using purified human cell extracts. In the course of the repair step, a label is incorporated in the DNA and this incorporation, which is a quantitative reflection of the number of repaired lesions, is subsequently revealed by chemiluminescence.

### 3) Methodology

Products Tested:

The products were assessed using the murine fibroblast line L929. The cells are pre-treated with the products (Table III) for 16 hours and are then stimulated with H₂O₂ (Hydrogen Peroxide 3%—Ref. GIFRER—Laboratoire Gifrer Barbezat) at 100 μM for 30 minutes.

TABLE III
Compilation of solutions tested

| Reference Product | Mother solution | Solutions tested |
|---|---|---|
| Unsaponifiable component of | 10 mg/ml | 3 μg/ml |
| pulp of fruit of the argan tree | (DMEM/ | |
| Batch: LK0304 | TWEEN20) | |
| | −20° C. | |
| Erythrodiol/EXTRASYNTHESE | 10 mg/ml DMSO | 3 μg/ml-6.78 μM |
| Batch: 05040605 | −20° C. | |
| Lupeol (triterpene) | 50 mM DMSO | 3 μg/ml-7 μM |
| | −20° C. | |
| α-Amyrine (triterpene) | 50 mM DMSO | 3 μg/ml-7 μM |
| | −20° C. | |
| β-Amyrine (triterpene) | 50 mM DMSO | 3 μg/ml-7 μM |
| | −20° C. | |

3D Test:

The principle is as follows: After damaging the genomic DNA (oxidative treatment), the cells are lysed. The lysate is deposited on a microplate coated with polylysine:
1—Adsorption of the DNA
2—Incubation of the DNA with a protein extract (enriched with repair enzymes) and a pool of nucleotides wherein one nucleotide is labeled with biotin (dUTP-Biotin)—Repair of the lesions and incorporation of the “dUTP-Biotin” labeled nucleotides in the DNA.
3—Incubation with an “Avidin-Peroxidase” enzyme complex—Recognition by the avidin of the incorporated dUTP-Biotin.
4—Addition of a luminescent peroxidase substrate and quantification of the emitted signal, which is proportional to the number of repaired lesions.

The test performance protocol is followed in accordance with the instructions of the kit supplier (Solyscel 3D Test—Ref: SFRIDN013— AES Laboratoire). At the end of the reaction, the plate is read in a luminometer (MITHRAS LB940-BERTHOLD).

Calculation of the Percentage DNA Protection:

The ratio below allows the percentage protection against the causation of lesions to DNA by oxidative stress to be calculated for each concentration of product tested (the Intensity of Luminescence—or IL—is an expression of the quantity of DNA lesions).

$$\% DNA protection = \frac{I L ( {H}_{2} {O}_{2} ) - I L ( product )}{I L ( {H}_{2} {O}_{2} ) - I L ( control )} \times 100$$

4) Results and conclusion

The results obtained in Example 3 showed that the anti-free-radical activity of erythrodiol is strongest at 3 μg/ml (6.78 μM). For that reason, the authors decided to test all the triterpenes (lupeol, α-amyrine, β-amyrine and erythrodiol) and the unsaponifiable extract of pulp of fruit of the argan tree at 3 μg/ml in the 3D “DNA Damage Detection” test. Said unsaponifiable extract was obtained in accordance with the method of Example 2.

The results obtained are compiled in Table IV below. The values indicated in this Table are percentages of inhibition (or % protection) of lesions to DNA following exogenous oxidative stress, compared to “basal control” cells (100%) and “H₂O₂ stressed” cells (0%).

TABLE IV
Protection of DNA by Erythrodiol

| | Intensity of | % lesions to | % DNA |
|---|---|---|---|
| | luminescence | DNA | protection |
| Control | 5460 | 0 | |
| H₂O₂ 100 μM-30 mins. | 11576.7 | 100 | 0 |
| Erythrodiol | 5520 | 1 | 99 |
| 3 μg/ml-6.78 μM | | | |
| Unsaponifiable | 6193.3 | 12 | 88 |
| component of pulp of | | | |
| fruit of the argan tree | | | |
| 3 μg/ml | | | |
| Lupeol | 9020 | 58.2 | 41.8 |
| 3 μg/ml-7 μM | | | |
| α-Amyrine | 8663.3 | 52.4 | 47.6 |
| 3 μg/ml-7 μM | | | |
| β-Amyrine | 13133.3 | 125.4 | −25.4 |
| 3 μg/ml-7 μM | | | |

The treatment with H₂O₂ brings about a marked proportion of oxidation at Guanine, with the formation of, in particular, 8-oxo-7,8-dihydro-2′-deoxyguanosine (8-OxoGuanine) (Dizdaroglu M. et al Arch. Biochem. Biophys. 1991; 285: 388-390). The 3D test shows a marked increase in luminescence after treatment with H₂O₂, reflecting marked repair activity and, consequently, a high level of damaged bases in the DNA.

The unsaponifiable extract of pulp of fruit of the argan tree is effective in protecting DNA from oxidative stress.

Erythrodiol, a compound contained in said unsaponifiable extract, at 3 μg/ml, has very good anti-oxidant activity, with 99% protection of the DNA from the formation of oxidative lesions.

When comparing the triterpene compounds at equivalent molar concentrations (about 7 μM), it is erythrodiol which is the most active.

### EXAMPLE 5

### In Vitro Study Model of the Effect of the Unsaponifiable Extract According to the Invention on the Induction of HSP72 Proteins

### 1) Bibliography

Various studies have shown the loss of inducibility of HSP72 proteins during ageing. In elderly patients, the induction of the HSP72 protein by heat is substantially reduced at the cutaneous level (Muramatsu T. et al. Br. J. Dermatol. 1996; 134: 1035-1038). Gustmann-Conrad A. et al. (Exp. Cell. Res. 1998; 241: 404-413) have moreover shown that induction of the HSP72 protein by thermal stress is significantly reduced in fibroblasts derived from the skin of elderly subjects, compared to those derived from young subjects. In that same study, it was shown that the level of induction of HSP72 is also reduced in fibroblasts (derived from young skin) or fibroblast lines (IMR-90) that have become senescent in the course of cell divisions.

A moderate first stress is sufficient, in vitro, to induce HSP proteins so that they will protect the cell against new stresses (Morris S. D. et al. J. Clin. Invest. 1996; 97: 706-12). HSP72 is a major protein of the HSP70 family, which is expressed in keratinocytes and cutaneous fibroblasts and is inducible by means of numerous stressing agents (heat, UV etc.) (Trautinger F. et al. J. Invest. Dermatol. 1993; 101: 334-38; Charveron M. et al. Cell. Biol. Toxicol. 1995; 11: 161-65).

### 2) Test Protocol

The authors of the present invention decided to analyze the level of induction, by means of thermal stress, of HSP72 proteins in IMR-90 fibroblasts (fibroblast line) during senescence, this being the case in order to assess the “anti-ageing” properties of an extract of pulp of fruit of the Argan tree, prepared according to Example 2 or, that is, containing 10% β-amyrine, 15% erythrodiol and 20% of the mixture lupeol/α-amyrine. Firstly, the authors established and validated a model of cellular ageing by inducing fibroblast senescence by means of oxidative stress.

Induced Senescence Model:

Fibroblasts divide until they reach a critical stage which is called replicative senescence and which is associated with cellular ageing. Senescence can, however, be induced, especially by oxidative stress, which is referred to as “Stress-induced Premature Senescence or SIPS” (Dumont et al Free Radic. Biol. Med. 2000; 28: 361-373). Model used: The induction of senescence in the young IMR-90 fibroblast line was brought about by treating the cells for 2 hours with H₂O₂. 72 hours after that stress, the IMR-90 cells are senescent.

Secondly, they demonstrated the reduction in the level of induction of HSP72 following thermal stress, in senescent fibroblasts compared to young fibroblasts. Finally, they assessed the properties of an extract of pulp of fruit of the Argan tree prepared in accordance with Example 2 or, that is, containing 10% β-amyrine, 15% erythrodiol and 20% of the mixture lupeol/α-amyrine, in this model of senescence.

### 3) Results

The invention will be better understood and the aims, advantages and characteristics thereof will emerge more clearly from the following description made with reference to the accompanying drawings, in which:

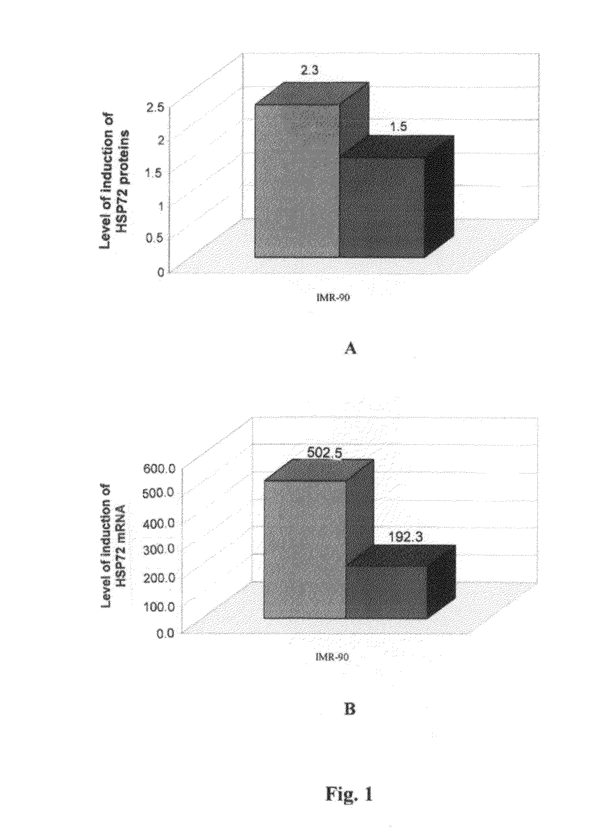

FIG. 1 shows the analysis of the level of induction of HSP72 in the IMR-90 fibroblasts at the level of transcription and translation;

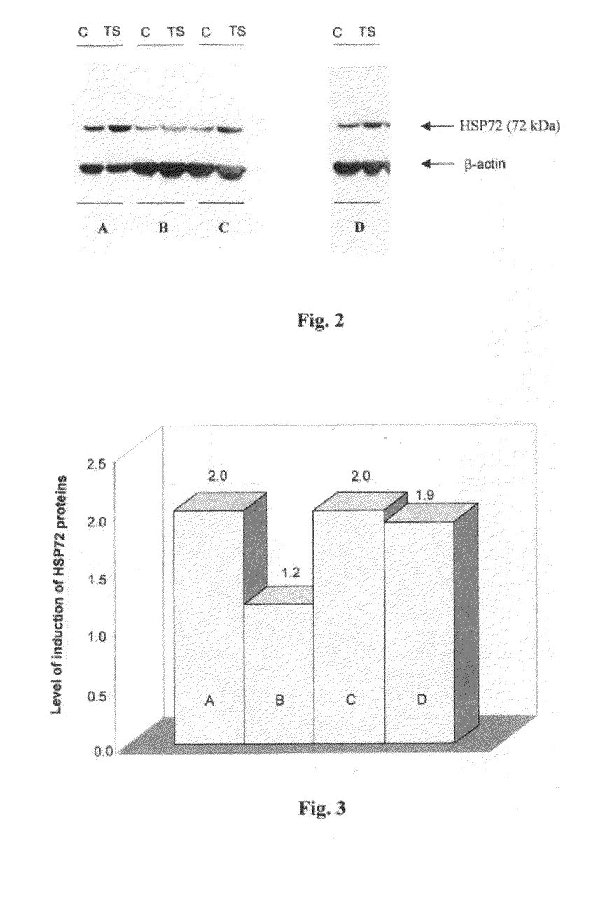

FIG. 2 shows the analysis, by Western Blot, of the level of HSP72 proteins in IMR-90 cells treated with different concentrations of the extract of pulp of fruit of the Argan tree in accordance with the invention;

FIG. 3 shows the semi-quantitative analysis of the level of induction of HSP72 proteins (normalized by the level of expression of β-actin) in senescent IMR-90 fibroblasts pre-treated with different concentrations of the extract of pulp of fruit of the Argan tree in accordance with the invention.

Analysis of the Induction of HSP72 by Thermal Stress in the Course of Senescence of IMR-90 Fibroblasts:

After being cultured at 37° C., the cells are incubated for 1 hour at 45° C. and are then incubated at 37° C. for 2 hours (mRNA analysis) or for 4 hours (protein analysis):

Protein Expression (Western Blot)

Having been extracted from the fibroblasts, the intracellular proteins were analyzed by the Western Blot technique, using an anti-HSP72 antibody (Monoclonal antibody, CHEMICON) and an indirect revelation system using luminescence. The membrane is analyzed and the intensity of the bands is quantified by densitometry (ImageMaster TotalLab software, AMERSHAM). The level of HSP72 expression is normalized by that of a constitutively expressed protein, β-actin.

FIG. 1A shows the semi-quantitative analysis, by Western Blot, of the level of induction of HSP72 proteins in young IMR-90 fibroblasts (▪) and senescent IMR-90 fibroblasts (▪) (senescence induced by H₂O₂). Accordingly, FIG. 1A clearly shows that the level of HSP72 proteins is induced by thermal stress in young IMR-90 fibroblasts. This induction of HSP72 is reduced in senescent IMR-90 fibroblasts.

mRNA Expression (Real-Time PCR)

The authors analyzed HSP72 expression at the level of transcription by quantifying the mRNA by the real-time PCR technique.

The level of expression of the gene of interest, HSP72, is calculated in samples treated with thermal stress and control samples. The level of expression of the HSP72 gene is then normalized by using three reference genes [β-actin, GAPDH (Human glyceraldehyde-3-phosphate dehydrogenase) and YWHAZ (Tyrosine-3-monooxygenase/tryptophan-5-monooxygenase activation protein, zeta polypeptide)], whose expression is constitutive.

Finally, by setting the level of expression in the control samples at 1, it is then possible to determine the HSP72 gene induction factor.

FIG. 1B shows the quantitative analysis, by real-time PCR, of the level of induction of HSP72 mRNA in young IMR-90 fibroblasts (▪) and senescent IMR-90 fibroblasts (▪) (senescence induced by H₂O₂). FIG. 1B clearly shows that, in the case of senescence induced in IMR-90 fibroblasts, the induction of HSP72 mRNA is also very reduced.

Analysis of the Efficacy of the Extract of Pulp of Fruit of the Argan Tree:

The authors of the invention used the model of induced senescence or SIPS (Stress Induced Premature Senescence) with the IMR-90 fibroblast line in order to assess the extract of pulp of fruit of the Argan tree, prepared in accordance with Example 2.

The cells were incubated with the extract of pulp of fruit of the Argan tree at concentrations of 1 and 3 μg/ml for 24 hours. They were then subjected to the senescence-inducing oxidative stress.

All the treatments were compared to a batch of “young” IMR90 cells and a batch of “senescent” (senescence-induced) cells not pre-treated with the extract of pulp of fruit of the Argan tree.

Three days (72 hours) after the oxidative stress, HSP72 proteins were induced by means of heat. Finally, the RNA and the HSP72 proteins were analyzed by real-time PCR and Western Blot, respectively.

FIG. 2 shows the analysis, by Western Blot, of the level of HSP72 proteins in the IMR-90 cells treated with different concentrations of the unsaponifiable extract prepared in accordance with Example 2. The legends “C” and “TS” signify “Control” and “Thermal Stress”, respectively. Analyses A, B, C and D relate to, respectively, young IMR-90 fibroblasts, senescent IMR-90 fibroblasts (senescence induced by H₂O₂), senescent IMR-90 fibroblasts incubated with the unsaponifiable extract at 1 μg/ml and, finally, senescent IMR-90 fibroblasts incubated with the unsaponifiable extract at 3 μg/ml. FIG. 3 shows the semi-quantitative analysis of the level of induction of HSP72 proteins (normalized by the level of expression of □-actin) in senescent IMR-90 fibroblasts pre-treated with the unsaponifiable extract at 1 μg/ml (C) and at 3 μg/ml (D). Also shown are the levels of induction of HSP72 proteins in young IMR-90 fibroblasts (A) and in senescent IMR-90 fibroblasts (B) (senescence induced by H₂O₂).

These FIGS. 2 and 3 show that there is no longer any induction of HSP72 proteins in IMR-90 fibroblasts that have become senescent, but that the extract of pulp of fruit of the Argan tree, at concentrations of 1 and 3 μg/ml, restores the induction of HSP72 by thermal stress.

Finally, Table V below gives the induction factor (after normalization) of HSP72 mRNA in senescent fibroblasts pre-treated with different concentrations of extract of pulp of fruit of the Argan tree.

TABLE V
Induction factor

| | Induction factor |
|---|---|
| Young IMR-90 | 109.1 |
| IMR-90, senescence induced by H₂O₂ | 79.0 |
| Senescent IMR-90 + extract of pulp of fruit of the | 84.3 |
| Argan tree 1 μg/ml | |
| Senescent IMR-90 + extract of pulp of fruit of the | 98.1 |
| Argan tree 3 μg/ml | |

This Table confirms the results obtained at the level of transcription and shows the almost total restoration of the induction of HSP72 mRNA by the extract of pulp of fruit of the Argan tree. It is at a concentration of 3 μg/ml that this extract is the most active.

### 4) Conclusion

HSP72 proteins are proteins which are inducible by means of numerous stresses (heat, etc.) and they play a major part in processes of adaptive response. It is recognized that the inducibility of HSP72 proteins, in the skin and in other tissues, reduces with age and especially in the case of cellular senescence. It is moreover accepted that ageing is associated with a reduction in the response to environmental stress, giving rise to age-related pathologies.

Starting from a model of senescence induced in fibroblasts in culture, the authors have assessed the capacity of the extract of pulp of fruit of the Argan tree to modulate the decrease in the induction of HSP72 by heat.

The results overall confirm, on the one hand, that there is a marked decrease in the induction of HSP72 (by thermal stress) in senescent fibroblasts compared to young fibroblasts. This work shows, on the other hand, that the extract of pulp of fruit of the Argan tree restores the induction of HSP72 proteins in senescent fibroblasts. In this in vitro study model, the extract of pulp of fruit of the Argan tree limits the biological consequences of cellular senescence and accordingly has anti-ageing properties.

1-13. (canceled)

14. A method for preventing and/or treating skin disorders associated with skin aging, comprising the step of administering an unsaponifiable extract of plant pulp, which extract comprises a triterpene fraction, which triterpene fraction comprises erythrodiol, α-amyrine, β-amyrine and lupeol, the amount of erythrodiol being between 7% and 40%, inclusive, by weight of the unsaponiflable extract, wherein the administration is effective for the prevention and/or treatment of the skin disorders.

15. The method of claim 14, wherein the mass fraction of β-amyrine is between 5% and 30%, inclusive, of the unsaponifiable extract.

16. The method of claim 14, wherein the sum of the mass fractions of α-amyrine and lupeol is between 10% and 50%, inclusive, of the unsaponifiable extract.

17. The method of claim 14, wherein the unsaponifiable extract of plant pulp is comprised in a cosmetic, pharmaceutical or nutraceutical product.

18. The method of claim 17, wherein the amount of the unsaponifiable extract in a final cosmetic product is between 0.001% and 50%, inclusive, by weight of the total weight of the product.

19. The method of claim 18, wherein the amount of the unsaponifiable extract in a final cosmetic product is between 0.01% and 10%, inclusive, by weight of the total weight of the product.

20. The method of claim 18, wherein the amount of the unsaponifiable extract in a final cosmetic product is between 0.1 and 2%, inclusive, by weight of the total weight of the product.

21. The method of claim 17, wherein the cosmetic, pharmaceutical or nutraceutical product is in an oral form or topical form.

22. The method of claim 21, wherein the topical form is selected from creams, gels, ointments and sprays.

23. The method of claim 21, wherein the oral form is selected from tablets, capsules, and powders for drinkable suspensions.

24. The method of claim 14, wherein the unsaponifiable extract is derived from a plant of the Sapotaceae botanical family.

25. The method of claim 14, wherein the unsaponifiable extract is derived from the pulp of the fruit of the Argan tree.

26. The method of claim 14, wherein the skin disorders are characterized by changes in the texture, color and transparency of the skin and by the appearance of wrinkles.

27. The method of claim 14, wherein the skin disorders are due to a reduction in, or loss of, response to environmental stress.

28. The method of claim 27, wherein the environmental stress is caused by sun exposure or tobacco exposure.

29. The method of claim 14, wherein the skin disorders are due to a reduction in, or loss of, the inducibility of HSP72 proteins.
